SEQUENCE LISTING (6 sequences)

SEQ ID NO: 1 - protein, 148 aa, Homo sapiens
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 2 - protein, 148 aa, Homo sapiens
AQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRS

SEQ ID NO: 3 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 399 aa, synthetic construct
MVGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 400 aa, synthetic construct
MVGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0338470 A1
(43) Pub. Date: Oct. 26, 2023

(54) METHODS AND COMPOSITIONS FOR TREATING OCULAR DISORDERS WITH FASR

(71) Applicant: ONL Therapeutics, Inc., Ann Arbor, MI (US)

(72) Inventor: Andrew J. KOCAB, Farmington Hills, MI (US)

(21) Appl. No.: 18/306,912

(22) Filed: Apr. 25, 2023

Related U.S. Application Data
(60) Provisional application No. 63/335,101, filed on Apr. 26, 2022.

(51) Int. Cl. A61K 38/17; A61K 9/00

(52) U.S. Cl. CPC A61K 38/177; A61K 9/0048

(57) ABSTRACT

A method of treating (e.g., inhibiting, reducing, preventing, etc.) inflammation in retinal tissue and/or a symptom thereof in an eye of an individual, the method comprising: administering a FasR protein to the eye, wherein the FasR protein reduces, inhibits, and/or prevents inflammation in the retinal tissue and/or the symptom thereof.

### CROSS-REFERENCE

This application claims benefit of U.S. Application No. 63/335,101, filed Apr. 26, 2022, which is herein incorporated by reference in its entirety.

### SEQUENCE LISTING

The instant application contains a Sequence Listing which has been submitted electronically in XML format and is hereby incorporated by reference in its entirety. The XML copy, created on Apr. 24, 2023, is named 58109715201.xml and is 7,683 bytes in size.

### BACKGROUND

Ocular disorders and diseases affect a substantive percentage of the world's population. The leading causes of blindness and low vision include primarily age-related eye diseases such as age-related macular degeneration, cataract, diabetic retinopathy, and glaucoma. However, more acute conditions also contribute to blindness and low vision. Treatment of eye conditions resulting in blindness or vision generally loss targets curing, as well as addressing, symptoms and progression. Treatment of chronic eye conditions further aims to prevent or delay the onset of irreversible vision impairment.

Ocular inflammation and its related complications are causes of vision loss. Vision is dependent on maintaining the integrity of the structure of the retina, and changes in retinal homeostasis resulting from retinal inflammation may provide the basis for vision loss and/or retinal disease. A number of conditions such as aging, metabolic abnormalities, altered vascular perfusion, or degenerative genetic conditions may also initiate various inflammatory processes within the retina. In the case of retinal inflammation, a dysregulated and/or prolonged immune response may contribute to both the pathogenesis of retinal diseases as well as vision threatening symptoms.

### SUMMARY

Despite advances in therapeutics aimed at treating ocular disorders and preserving or improving vision, the burden resulting from ocular diseases and disorders continues to increase. Many challenges exist in the development of therapeutics for treating disease and disorders in the eye of an individual.

Described and provided herein are methods of treating (e.g., inhibiting, reducing, preventing, etc.) inflammation in retinal tissue and/or a symptom thereof in an eye of an individual, the method comprising: administering a FasR protein to the eye, wherein the FasR protein reduces, inhibits, and/or prevents inflammation in the retinal tissue and/or the symptom thereof.

Also described and provided herein are methods of treating (e.g., inhibiting, reducing, preventing, etc.) retinal cell loss (e.g., retinal cell death) and/or a symptom thereof in an eye of an individual, the method comprising: administering a FasR protein to the eye, wherein the FasR protein reduces, inhibits, and/or prevents retinal cell loss and/or the symptom thereof.

In some embodiments, the retinal cells comprise pigment epithelial cells, retinal ganglion cells, photoreceptors, or a combination thereof.

Described and provided herein are methods of treating an ocular disorder or a symptom thereof, in an eye of an individual, the method comprising: administering a FasR protein to the eye, thereby treating the ocular disorder or symptom thereof.

In some embodiments, the ocular disorder comprises retinal degeneration. In some embodiments, the ocular disorder comprises glaucoma. In some embodiments, the ocular disorder comprises macular degeneration. In some embodiments, the ocular disorder comprises a retinal detachment. In some embodiments, the ocular disorder comprises inherited retinal degeneration (e.g., retinitis pigmentosa). In some embodiments, the ocular disorder comprises a vascular disease (e.g., retinal arterial occlusive disease or central retinal vein occlusion).

In some embodiments, the FasR protein comprises a FasR monomer, a FasR dimer, or a FasR trimer. In some embodiments, the FasR protein is fused to a second protein. In some embodiments, the second protein extends the half-life of the FasR protein. In some embodiments, the second protein comprises an Fc protein. In some embodiments, the second protein comprises a variant Fc protein comprising one or more mutations relative to a wildtype Fc region, wherein the variant Fc region exhibits reduced effector function compared to the wildtype Fc region. In some embodiments, the reduced effector function is selected from the list consisting of reduced antibody-dependent cell-mediated cytotoxicity (ADCC), reduced complement mediated cytotoxicity (CDC), reduced affinity for C1q, and any combination thereof. In some embodiments, the variant Fc protein comprises IgG1 Fc region, and wherein the one or more mutations comprises (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (I) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(uu), per EU numbering. In some embodiments, the second protein comprises a fucosylated Fc protein. In some embodiments, the FasR protein is fully or partially glycosylated.

Described and provided herein are methods of treating (e.g., inhibiting, reducing, preventing, etc.) inflammation in retinal tissue and/or a symptom thereof in an eye of an individual, the method comprising: administering a polynucleic acid molecule comprising a sequence encoding a FasR protein to the eye, wherein the FasR protein reduces, inhibits, and/or prevents inflammation in the retinal tissue and/or the symptom thereof. Also described and provided herein are methods of treating (e.g., inhibiting, reducing, preventing, etc.) retinal cell loss and/or a symptom thereof in an eye of an individual, the method comprising: administering a polynucleic acid molecule comprising a sequence encoding a FasR protein to the eye, wherein the FasR protein reduces, inhibits, and/or prevents retinal cell loss and/or the symptom thereof. In some embodiments, the retinal cells comprise pigment epithelial cells, retinal ganglion cells, photoreceptors, or a combination thereof.

Described and provided herein are methods of treating an ocular disorder or a symptom thereof, in an eye of an individual, the method comprising: administering a polynucleic acid molecule comprising a sequence encoding a FasR protein to the eye, thereby treating the ocular disorder or symptom thereof.

In some embodiments, the ocular disorder comprises retinal degeneration. In some embodiments, the ocular disorder comprises glaucoma. In some embodiments, the ocular disorder comprises macular degeneration. In some embodiments, the ocular disorder comprises a retinal detachment. In some embodiments, the ocular disorder comprises inherited retinal degeneration (e.g., inherited retinal degeneration (e.g., retinitis pigmentosa)). In some embodiments, the ocular disorder comprises a vascular disease (e.g., retinal arterial occlusive disease or central retinal vein occlusion).

In some embodiments, the polynucleic acid molecule encodes the FasR protein fused to a second protein. In some embodiments, the second protein extends the half-life of the FasR protein.

In some embodiments, the second protein comprises an Fc protein.

In some embodiments, the second protein comprises a variant Fc protein comprising one or more mutations relative to a wildtype Fc region, wherein the variant Fc region exhibits reduced effector function compared to the wildtype Fc region. In some embodiments, the reduced effector function is selected from the list consisting of reduced antibody-dependent cell-mediated cytotoxicity (ADCC), reduced complement mediated cytotoxicity (CDC), reduced affinity for C1q, and any combination thereof. In some embodiments, the variant Fc protein comprises IgG1 Fc region, and wherein the one or more mutations comprises (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (I) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(uu), per EU numbering.

In some embodiments, the polynucleic acid molecule further comprises a promoter sequence, and wherein the promoter sequence drives transcription of the sequence encoding the FasR protein. In some embodiments, the promoter is a ubiquitous promoter. In some embodiments, the promoter is a tissue-specific promoter. In some embodiments, the nucleic acid is administered within a vector, wherein the vector is a lipid nanoparticle (e.g., a liposome) or a viral vector (e.g., AAV or lentivirus).

Further described and provided herein are fusion proteins comprising a FasR protein and a dead Fc protein (e.g., a variant Fc comprising one or more mutations relative to a wildtype Fc region, and wherein the variant Fc region exhibits reduced effector function compared to the wildtype Fc region).

In some embodiments, the dead Fc protein comprises one or more mutations relative to a wildtype Fc region that reduce effector function (e.g., antibody-dependent cell-mediated cytotoxicity (ADCC), reduced complement mediated cytotoxicity (CDC), reduced affinity for C1q, and any combination thereof) compared to the wildtype Fc region. In some embodiments, the dead Fc protein comprises IgG1 Fc region, and wherein the one or more mutations comprises (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (I) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311 E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (Ill) A330L, (mmm) P331A or P331S, or (nnn) E233P, (ooo) L234A, L235E, G237A, A330S, and P331S or (ppp) any combination of (a)-(uu), per EU numbering.

In some embodiments, the dead Fc protein comprises one or more of the mutations in Table 1.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic representation of exemplary CD95 molecules.

### DETAILED DESCRIPTION

Provided herein are provided herein are methods that are advantageous in that the methods, generally, utilize administering a FasR protein to inhibit, reduce, and/or prevent cellular FasR signaling (e.g., by binding to FasL and preventing an interaction between cellular FasR and FasL). In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye. In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye. Exemplary inflammatory molecules include, but are not limited to, Fas-mediated inflammation-related molecules (e.g. TNFa, IL-1 b, IP-10, IL-18, MIPI a, IL-6, GFAP, MIP2, MCP-1, or MIP-1 b); a Fas-mediated complement-related molecules (complement component 3 (C3) or complement component Iq (CIq)) Caspase 8; components of the inflammasome (e.g., NLRP3 or NLRP2); C-X-C motif chemokines (e.g., CXCL2 (MIP-2alpha) or CXCL10 (IP-10)); C-X3-C motif chemokines (e.g., CX3CL1 (fractalkine)); C-C motif chemokines (CCL2 (MCP-1), CCL3 (MIP-1 a), and CCL4 (MIP-1 b)); toll-like receptor 4 (TLR4); interleukin cytokines (e.g., IL-1 b, IL-18, and IL-6); TNF superfamily cytokines (e.g., TNFa); or GFAP. In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye).

Provided and described herein are methods of treating (e.g., inhibiting, reducing, preventing, etc.) inflammation in retinal tissue and/or a symptom thereof in an eye of an individual, the method comprising: administering a FasR protein to the eye, wherein the FasR protein reduces, inhibits, and/or prevents inflammation in the retinal tissue and/or the symptom thereof.

Also provided and described herein are methods of treating (e.g., inhibiting, reducing, preventing, etc.) retinal cell loss and/or a symptom thereof in an eye of an individual, the method comprising: administering a FasR protein to the eye, wherein the FasR protein reduces, inhibits, and/or prevents retinal cell loss and/or the symptom thereof. In some embodiments, the retinal cells comprise pigment epithelial cells, retinal ganglion cells, photoreceptors, or a combination thereof. In some embodiments, the retinal cells comprise pigment epithelial cells. In some embodiments, the retinal cells comprise retinal ganglion cells. In some embodiments, the retinal cells comprise photoreceptors.

In some embodiments, treating (1) inflammation in the eye, (2) retinal cell loss, and/or (3) retinal degeneration is useful for treating an ocular disease, disorder, or condition. In another embodiment, inhibiting, reducing, and/or preventing (1) inflammation in the eye, (2) retinal cell loss, and/or (3) retinal degeneration is useful for treating, inhibiting, reducing the pathology of, and/or preventing an ocular disease, disorder, or condition. As used herein, ocular disease, disorder, or condition generally includes and/or refers to a disorder or pathological condition of the eye which is not normal to an eye in a healthy state.

Further provided and described here are methods of treating an ocular disorder or a symptom thereof, in an eye of an individual, the method comprising: administering a FasR protein to the eye, thereby treating the ocular disorder or symptom thereof. In some embodiments, the ocular disorder comprises retinal degeneration. In some embodiments, the ocular disorder comprises glaucoma. In some embodiments, the ocular disorder comprises macular degeneration. In some embodiments, the ocular disorder comprises a retinal detachment. In some embodiments, the ocular disorder comprises inherited retinal degeneration (e.g., retinitis pigmentosa). In some embodiments, the ocular disorder comprises a vascular disease (e.g., retinal arterial occlusive disease or central retinal vein occlusion).

In some embodiments, the FasR protein is fused to a second protein (e.g., a FasR fusion protein). In some embodiments, the second protein extends the half-life of the FasR protein. In some embodiments, the second protein comprises an Fc protein. In some embodiments, the second protein comprises a variant Fc protein comprising one or more mutations relative to a wildtype Fc region, wherein the variant Fc region exhibits reduced effector function compared to the wildtype Fc region. In some embodiments, the reduced effector function is selected from the list consisting of reduced antibody-dependent cell-mediated cytotoxicity (ADCC), reduced complement mediated cytotoxicity (CDC), reduced affinity for C1q, and any combination thereof.

The FasR protein can also be provided by administering a polynucleic acid molecule comprising a sequence encoding the FasR protein. Provided and described method of treating (e.g., inhibiting, reducing, preventing, etc.) inflammation in retinal tissue and/or a symptom thereof in an eye of an individual, the method comprising: administering a polynucleic acid molecule comprising a sequence encoding a FasR protein to the eye, wherein the FasR protein reduces, inhibits, and/or prevents inflammation in the retinal tissue and/or the symptom thereof. Also provided and described are methods of treating (e.g., inhibiting, reducing, preventing, etc.) retinal cell loss and/or a symptom thereof in an eye of an individual, the method comprising: administering a polynucleic acid molecule comprising a sequence encoding a FasR protein to the eye, wherein the FasR protein reduces, inhibits, and/or prevents retinal cell loss and/or the symptom thereof. Further provided and described are methods of treating an ocular disorder or a symptom thereof, in an eye of an individual, the method comprising: administering a polynucleic acid molecule comprising a sequence encoding a FasR protein to the eye, thereby treating the ocular disorder or symptom thereof.

In some embodiments, the polynucleic acid molecule comprises a sequence encoding the FasR protein fused to a second protein. In some embodiments, the second protein comprises an Fc protein. In some embodiments, the second protein comprises a variant Fc protein comprising one or more mutations relative to a wildtype Fc region, wherein the variant Fc region exhibits reduced effector function compared to the wildtype Fc region. In some embodiments, the reduced effector function is selected from the list consisting of reduced antibody-dependent cell-mediated cytotoxicity (ADCC), reduced complement mediated cytotoxicity (CDC), reduced affinity for C1q, and any combination thereof.

In some embodiments, the polynucleic acid molecule further comprises a promoter sequence, and wherein the promoter sequence drives transcription of the sequence encoding the FasR protein. In some embodiments, the promoter is a ubiquitous promoter. In some embodiments, the promoter is a tissue-specific promoter. In some embodiments, the nucleic acid is administered within a vector, wherein the vector is a lipid nanoparticle (e.g., a liposome) or a viral vector (e.g., AAV or lentivirus).

### FasR Proteins

The term “Fas receptor”, “FasR”, “Tumor necrosis factor receptor superfamily member 6”, “TNFRSF6”, “CD95 Antigen”, “CD95” or “TNFR6” are used synonymously herein and generally include and/or refer to receptor for which pertinent information can be retrieved from UniProt under the identifier P25445 or NCBI gene ID: 355. The protein is a member of the TNF-receptor superfamily. FasR has been shown to play a central role in the physiological regulation of programmed cell death and has been implicated in the pathogenesis of various malignancies and diseases of the immune system. The interaction of this receptor with its ligand (FAS ligand or FasL) allows the formation of a death-inducing signaling complex that includes Fas-associated death domain protein (FADD), caspase 8, and caspase 10. The autoproteolytic processing of the caspases in the complex triggers a downstream caspase cascade and leads to apoptosis. This receptor has been also shown to activate NF-kappaB, MAPK3/ERK1, and MAPK8/JNK, and is found to be involved in transducing the proliferating signals in normal diploid fibroblast and T cells.

The term “FAS ligand” or “Tumour Necrosis Factor Ligand Superfamily Member 6”, “TNFSF6”, “TNFR6 ligand”, “CD95L” or “CD95 ligand” are used synonymously herein and generally include and refer to a protein which is a member of the tumour necrosis factor superfamily that binds to TNFRSF6/FAS, a receptor that transduces the apoptotic signal into cells. Pertinent information can be derived from the UniProt database under the accession number P48023 or NCBI gene ID: 256. The primary function of the encoded transmembrane protein is the induction of apoptosis triggered by binding to FAS receptor. The FAS/FASLG signaling pathway is important for immune system regulation, including activation-induced cell death (AICD) of T cells and cytotoxic T lymphocyte induced cell death. The FAS/FASLG signaling pathway has also been shown to be important in the regulation of cell death across multiple cell types (e.g., non-immune cells) It has also been implicated in the progression of several cancers. Defects in this gene may be related to some cases of systemic lupus erythematosus (SLE). Alternatively spliced transcript variants have been described.

The extracellular domain of FasR is useful for the FasR proteins provided and described herein. In some embodiment, the FasR protein comprises the extracellular domain of FasR (e.g., amino acids 26-173 of FasR, SEQ ID NO: 1, or SEQ ID NO: 2). In some embodiments, the FasR protein comprises an amino acid sequence having about 80% sequence identity to SEQ ID NO: 1 to about 100% sequence identity to SEQ ID NO: 1. In some embodiments, the FasR protein comprises an amino acid sequence having about 80% sequence identity to SEQ ID NO: 1 to about 85% sequence identity to SEQ ID NO: 1, about 80% sequence identity to SEQ ID NO: 1 to about 90% sequence identity to SEQ ID NO: 1, about 80% sequence identity to SEQ ID NO: 1 to about 95% sequence identity to SEQ ID NO: 1, about 80% sequence identity to SEQ ID NO: 1 to about 97% sequence identity to SEQ ID NO: 1, about 80% sequence identity to SEQ ID NO: 1 to about 98% sequence identity to SEQ ID NO: 1, about 80% sequence identity to SEQ ID NO: 1 to about 99% sequence identity to SEQ ID NO: 1, about 80% sequence identity to SEQ ID NO: 1 to about 100% sequence identity to SEQ ID NO: 1, about 85% sequence identity to SEQ ID NO: 1 to about 90% sequence identity to SEQ ID NO: 1, about 85% sequence identity to SEQ ID NO: 1 to about 95% sequence identity to SEQ ID NO: 1, about 85% sequence identity to SEQ ID NO: 1 to about 97% sequence identity to SEQ ID NO: 1, about 85% sequence identity to SEQ ID NO: 1 to about 98% sequence identity to SEQ ID NO: 1, about 85% sequence identity to SEQ ID NO: 1 to about 99% sequence identity to SEQ ID NO: 1, about 85% sequence identity to SEQ ID NO: 1 to about 100% sequence identity to SEQ ID NO: 1, about 90% sequence identity to SEQ ID NO: 1 to about 95% sequence identity to SEQ ID NO: 1, about 90% sequence identity to SEQ ID NO: 1 to about 97% sequence identity to SEQ ID NO: 1, about 90% sequence identity to SEQ ID NO: 1 to about 98% sequence identity to SEQ ID NO: 1, about 90% sequence identity to SEQ ID NO: 1 to about 99% sequence identity to SEQ ID NO: 1, about 90% sequence identity to SEQ ID NO: 1 to about 100% sequence identity to SEQ ID NO: 1, about 95% sequence identity to SEQ ID NO: 1 to about 97% sequence identity to SEQ ID NO: 1, about 95% sequence identity to SEQ ID NO: 1 to about 98% sequence identity to SEQ ID NO: 1, about 95% sequence identity to SEQ ID NO: 1 to about 99% sequence identity to SEQ ID NO: 1, about 95% sequence identity to SEQ ID NO: 1 to about 100% sequence identity to SEQ ID NO: 1, about 97% sequence identity to SEQ ID NO: 1 to about 98% sequence identity to SEQ ID NO: 1, about 97% sequence identity to SEQ ID NO: 1 to about 99% sequence identity to SEQ ID NO: 1, about 97% sequence identity to SEQ ID NO: 1 to about 100% sequence identity to SEQ ID NO: 1, about 98% sequence identity to SEQ ID NO: 1 to about 99% sequence identity to SEQ ID NO: 1, about 98% sequence identity to SEQ ID NO: 1 to about 100% sequence identity to SEQ ID NO: 1, or about 99% sequence identity to SEQ ID NO: 1 to about 100% sequence identity to SEQ ID NO: 1. In some embodiments, the FasR protein comprises an amino acid sequence having about 80% sequence identity to SEQ ID NO: 1, about 85% sequence identity to SEQ ID NO: 1, about 90% sequence identity to SEQ ID NO: 1, about 95% sequence identity to SEQ ID NO: 1, about 97% sequence identity to SEQ ID NO: 1, about 98% sequence identity to SEQ ID NO: 1, about 99% sequence identity to SEQ ID NO: 1, or about 100% sequence identity to SEQ ID NO: 1. In some embodiments, the FasR protein comprises an amino acid sequence having at least about 80% sequence identity to SEQ ID NO: 1, about 85% sequence identity to SEQ ID NO: 1, about 90% sequence identity to SEQ ID NO: 1, about 95% sequence identity to SEQ ID NO: 1, about 97% sequence identity to SEQ ID NO: 1, about 98% sequence identity to SEQ ID NO: 1, or about 99% sequence identity to SEQ ID NO: 1.

In some embodiments, the FasR protein comprises an amino acid sequence having about 80% sequence identity to SEQ ID NO: 2 to about 100% sequence identity to SEQ ID NO: 2. In some embodiments, the FasR protein comprises an amino acid sequence having about 80% sequence identity to SEQ ID NO: 2 to about 85% sequence identity to SEQ ID NO: 2, about 80% sequence identity to SEQ ID NO: 2 to about 90% sequence identity to SEQ ID NO: 2, about 80% sequence identity to SEQ ID NO: 2 to about 95% sequence identity to SEQ ID NO: 2, about 80% sequence identity to SEQ ID NO: 2 to about 97% sequence identity to SEQ ID NO: 2, about 80% sequence identity to SEQ ID NO: 2 to about 98% sequence identity to SEQ ID NO: 2, about 80% sequence identity to SEQ ID NO: 2 to about 99% sequence identity to SEQ ID NO: 2, about 80% sequence identity to SEQ ID NO: 2 to about 100% sequence identity to SEQ ID NO: 2, about 85% sequence identity to SEQ ID NO: 2 to about 90% sequence identity to SEQ ID NO: 2, about 85% sequence identity to SEQ ID NO: 2 to about 95% sequence identity to SEQ ID NO: 2, about 85% sequence identity to SEQ ID NO: 2 to about 97% sequence identity to SEQ ID NO: 2, about 85% sequence identity to SEQ ID NO: 2 to about 98% sequence identity to SEQ ID NO: 2, about 85% sequence identity to SEQ ID NO: 2 to about 99% sequence identity to SEQ ID NO: 2, about 85% sequence identity to SEQ ID NO: 2 to about 100% sequence identity to SEQ ID NO: 2, about 90% sequence identity to SEQ ID NO: 2 to about 95% sequence identity to SEQ ID NO: 2, about 90% sequence identity to SEQ ID NO: 2 to about 97% sequence identity to SEQ ID NO: 2, about 90% sequence identity to SEQ ID NO: 2 to about 98% sequence identity to SEQ ID NO: 2, about 90% sequence identity to SEQ ID NO: 2 to about 99% sequence identity to SEQ ID NO: 2, about 90% sequence identity to SEQ ID NO: 2 to about 100% sequence identity to SEQ ID NO: 2, about 95% sequence identity to SEQ ID NO: 2 to about 97% sequence identity to SEQ ID NO: 2, about 95% sequence identity to SEQ ID NO: 2 to about 98% sequence identity to SEQ ID NO: 2, about 95% sequence identity to SEQ ID NO: 2 to about 99% sequence identity to SEQ ID NO: 2, about 95% sequence identity to SEQ ID NO: 2 to about 100% sequence identity to SEQ ID NO: 2, about 97% sequence identity to SEQ ID NO: 2 to about 98% sequence identity to SEQ ID NO: 2, about 97% sequence identity to SEQ ID NO: 2 to about 99% sequence identity to SEQ ID NO: 2, about 97% sequence identity to SEQ ID NO: 2 to about 100% sequence identity to SEQ ID NO: 2, about 98% sequence identity to SEQ ID NO: 2 to about 99% sequence identity to SEQ ID NO: 2, about 98% sequence identity to SEQ ID NO: 2 to about 100% sequence identity to SEQ ID NO: 2, or about 99% sequence identity to SEQ ID NO: 2 to about 100% sequence identity to SEQ ID NO: 2. In some embodiments, the FasR protein comprises an amino acid sequence having about 80% sequence identity to SEQ ID NO: 2, about 85% sequence identity to SEQ ID NO: 2, about 90% sequence identity to SEQ ID NO: 2, about 95% sequence identity to SEQ ID NO: 2, about 97% sequence identity to SEQ ID NO: 2, about 98% sequence identity to SEQ ID NO: 2, about 99% sequence identity to SEQ ID NO: 2, or about 100% sequence identity to SEQ ID NO: 2. In some embodiments, the FasR protein comprises an amino acid sequence having at least about 80% sequence identity to SEQ ID NO: 2, about 85% sequence identity to SEQ ID NO: 2, about 90% sequence identity to SEQ ID NO: 2, about 95% sequence identity to SEQ ID NO: 2, about 97% sequence identity to SEQ ID NO: 2, about 98% sequence identity to SEQ ID NO: 2, or about 99% sequence identity to SEQ ID NO: 2.

Also included are truncations of the Fas R proteins described herein. As used herein, the term “fragment” generally designates a “functional fragment”, e.g., a fragment or portion of a wild-type or full-length protein which has essentially the same biological activity and/or properties as the corresponding wild-type or full-length protein (e.g., as measured by ELISA binding to FasL).

In some embodiments, the FasR comprises a N-terminus truncation. In certain embodiments, the N-terminus truncation comprise -1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 and/or 50 N-terminally shortened variants of SEQ ID NO: 5 or 6. In certain embodiments, the N-terminus truncation comprises 16, 20, or 25 N-terminally shortened variants. The numbering refers to the APG101 protein including signal sequence according to SEQ ID NO: 1.

In some embodiments, the FasR protein comprises a monomer, a dimer, or a trimer. In certain embodiments, the FasR protein comprises a FasR protein monomer. In certain embodiments, the FasR protein comprises a FasR protein dimer. In certain embodiments, the FasR protein comprises a FasR protein trimer. Methods for designing and constructing FasR dimer and trimers are known within the art. For example, a trimerization polypeptide can be employed to promote trimerization of the FasR protein. Exemplary trimerization polypeptides include, collectin trimerization domains and/or polypeptides. Collectin trimerization domains and/or polypeptides as used herein is generally derived from the C-terminal part of Collectin polypeptides. The trimerization domain as used herein comprises a coiled-coil region (in certain embodiments referred to as neck region) and a Carbohydrate Recognition Domain (referred to herein also as CRD). The collectin trimerization domain may comprise any collectin family member. Such members and their structures are summarized in, e.g., Hakansson et al. (Protein Science, 2000, 9:1607-1617) and may comprise surfactant protein-D (acc. No.: P35247), surfactant protein-A 1 (acc. No.: Q8IWL2), surfactant protein-A 2 (acc. No.: Q8IWL1), mannan-binding-protein-C (accession No.: P11226), collectin liver 1 (acc. No.: Q9Y6Z7), collectin placenta 1 (acc. No.: Q5KU26), or collectin-11 (acc. No.: Q9BWP8). As well the coiled-coil region (neck region) as the CRD may be selected from the above mentioned collectins. It must be understood that coiled-coil (neck region) and CRD may but need not be from the same collectin.

A polypeptide or protein is used interchangeably, and generally encompasses and/or refers to a polymer of amino acid residues and is not limited to a minimum length. Proteins, including the provided fusion proteins and fusion polypeptide chains and other peptides, e.g., linkers and binding peptides, can include amino acid residues including natural and/or non-natural amino acid residues. The terms also include post-expression modifications of the protein, for example, glycosylation, sialylation, acetylation, phosphorylation, and the like. In some embodiments, the proteins can contain modifications with respect to a native or natural sequence, as long as the protein maintains the desired activity. These modifications can be deliberate, as through site-directed mutagenesis, or can be accidental, for example, through mutations of hosts which produce the proteins or errors due to PCR amplification.

The term recombinant indicates that the material (e.g., a nucleic acid or a protein) has been artificially or synthetically (e.g., non-naturally) altered by human intervention. The alteration can be performed on the material within, or removed from, its natural environment or state. For example, a recombinant nucleic acid is one that is made by recombining nucleic acids, e.g., during cloning, DNA shuffling, or other well-known molecular biological procedures. A recombinant DNA molecule is comprised of segments of DNA joined together by means of such molecular biological techniques. The term recombinant protein or recombinant protein as used herein refers to a protein molecule which is expressed using a recombinant DNA molecule. A recombinant host cell is a cell that contains and/or expresses a recombinant nucleic acid.

A polynucleotide sequence or nucleotide sequence or nucleic acid sequence, as used interchangeably herein, is a polymer of nucleotides, including an oligonucleotide, a DNA, and RNA, a nucleic acid, or a character string representing a nucleotide polymer, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand.

Polynucleotide and protein sequences of the current disclosure can be defined in terms of particular identity and/or similarity with certain polynucleotides and proteins described herein. In some embodiments, sequence identity will typically be greater than 60%, greater than 75%, greater than 80%, greater than 90%, and/or greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence disclosed herein. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAST, Novoalign (Novocraft Technologies, ELAND (Illumina, San Diego, Calif.), SOAP (available at soap.genomics.org.cn), and Maq (available at maq.sourceforge.net). In some embodiments, BLAST searches are be performed with the BLAST program, score=100, wordlength=12, to obtain sequences with the desired percent sequence identity. In some embodiments, to obtain gapped alignments for comparison purposes, Gapped BLAST. When utilizing BLAST and Gapped BLAST programs, the default parameters of the respective programs (NBLAST and XBLAST) can be used. In some embodiments, Clustal Omega is used.

As used herein, the terms nucleic acid molecule encoding, DNA sequence encoding, nucleic acid sequence encoding, or DNA encoding refer to the order or sequence of deoxyribonucleotides along a strand of deoxyribonucleic acid. The order of these deoxyribonucleotides determines the order of ribonucleotides along the mRNA chain, and also determines the order of amino acids along the protein (protein) chain. The DNA sequence thus codes for the RNA sequence and for the amino acid sequence.

Expression of a gene or expression of a nucleic acid means transcription of DNA into RNA (optionally including modification of the RNA, e.g., splicing), translation of RNA into a protein (possibly including subsequent post-translational modification of the protein), or both transcription and translation, as indicated by the context.

The term gene is used broadly to refer to any nucleic acid associated with a biological function. Genes typically include coding sequences and/or the regulatory sequences required for expression of such coding sequences. The term gene applies to a specific genomic or recombinant sequence, as well as to a cDNA or mRNA encoded by that sequence. A fusion gene contains a coding region that encodes a transgene. Genes also include non-expressed nucleic acid segments that, for example, form recognition sequences for other proteins. Non-expressed regulatory sequences including transcriptional control elements to which regulatory proteins, for example, transcription factors, bind, resulting in transcription of adjacent or nearby sequences.

As used herein the term coding region or coding sequence when used in reference to a structural gene refers to the nucleotide sequences which encode the amino acids found in the nascent protein as a result of translation of an mRNA molecule. The coding region is bounded, in eukaryotes, on the 5′ side by the nucleotide triplet ATG which encodes the initiator methionine and on the 3′ side by one of the three triplets which specify stop codons (e.g., TAA, TAG, TGA). Transcriptional control signals in eukaryotes comprise promoter and enhancer elements. Promoters and enhancers consist of short arrays of DNA sequences that interact specifically with cellular proteins involved in transcription. Promoter and enhancer elements have been isolated from a variety of eukaryotic sources including genes in yeast, insect, and mammalian cells and viruses (analogous control elements, e.g., promoters, are also found in prokaryotes). The selection of a particular promoter and enhancer depends on what cell type is to be used to express the protein of interest. Some eukaryotic promoters and enhancers have a broad host range while others are functional in a limited subset of cell types.

The term expression vector refers to a recombinant DNA molecule containing a desired coding sequence and appropriate nucleic acid sequences necessary for the expression of the operably linked coding sequence in a particular host cell. Nucleic acid sequences necessary for expression in prokaryotes include a promoter, optionally an operator sequence, a ribosome binding site, and possibly other sequences. Eukaryotic cells are known to utilize promoters, enhancers, and termination and polyadenylation signals. A secretory signal peptide sequence can also, optionally, be encoded by the expression vector, operably linked to the coding sequence for the inventive toxin peptide analog, so that the expressed toxin peptide analog can be secreted by the recombinant host cell, for more facile isolation of the toxin peptide analog from the cell, if desired. Such techniques are well known in the art.

In certain embodiments, the protein or protein or fusion protein comprises one or more naturally occurring amino acids. In certain embodiments, the protein or fusion protein consists of naturally occurring amino acids. As used herein, naturally occurring amino acids include and/or refer to amino acids which are generally found in nature and are not manipulated by man. In certain instances, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the protein or fusion protein comprises a variant sequence of the protein or fusion protein. In certain instances, amino acid substitutions can be made in the sequence of any of the protein or fusion proteins described herein, without necessarily decreasing or ablating its activity (as measured by, e.g., the binding or functional assays described herein). Accordingly, in some embodiments, the variant sequence comprises one or more amino acid substitutions. In certain embodiments, the variant sequence comprises one amino acid substitution. In certain embodiments, the variant sequence comprises two amino acid substitutions. In certain embodiments, the variant sequence comprises three amino acid substitutions. In certain instances, substitutions include conservative substitutions (e.g., substitutions with amino acids of comparable chemical characteristics). In certain instances, a non-polar amino acid can be substituted and replaced with another non-polar amino acid, wherein non-polar amino acids include alanine, leucine, isoleucine, valine, glycine, proline, phenylalanine, tryptophan, and methionine. In certain instances, a neutrally charged polar amino acids can be substituted and replaced with another neutrally charged polar amino acid, wherein neutrally charged polar amino acids include serine, threonine, cysteine, tyrosine, asparagine, and glutamine. In certain instances, a positively charged amino acid can be substituted and replaced with another positively charged amino acid, wherein positively charged amino acids include arginine, lysine, and histidine. In certain instances, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In certain embodiments, the protein or fusion protein comprises one or more non-natural amino acids. In certain embodiments, the protein or fusion protein consists of non-natural amino acids. As used herein, non-natural amino acids and/or unnatural amino acids include and/or refer to amino acid structures that cannot be generated biosynthetically in any organism using unmodified or modified genes from any organism. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), Naphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring protein or fusion protein sequence. A non-natural amino acid residue also can be incorporated such that a desired functionality is imparted to the apelin molecule, for example, the ability to link a functional moiety (e.g., PEG).

An isolated nucleic acid generally encompasses and/or refers to a nucleic acid molecule that has been separated from a component of its natural environment. An isolated nucleic acid encompasses a nucleic acid molecule contained in cells that ordinarily contain the nucleic acid molecule, but the nucleic acid molecule is present extrachromosomally or at a chromosomal location that is different from its natural chromosomal location. An Isolated nucleic acid encoding a protein or fusion protein encompasses and/or refers to one or more nucleic acid molecules encoding the protein or fusion protein (or fragments thereof), including such nucleic acid molecule(s) in a single vector or separate vectors, and such nucleic acid molecule(s) present at one or more locations in a host cell. In some embodiments, provided herein is a nucleic acid molecule encoding any of the protein or fusion proteins described herein

A pharmaceutical formulation generally encompasses and/or refers to a preparation which is in such form as to permit the biological activity of an active ingredient contained therein to be effective, and which contains no additional components which are unacceptably toxic to a subject to which the formulation would be administered.

A pharmaceutically acceptable carrier generally encompasses and/or refers to an ingredient in a pharmaceutical formulation, other than an active ingredient, which is nontoxic to a subject. A pharmaceutically acceptable carrier encompasses, but is not limited to, a buffer, excipient, stabilizer, or preservative.

### FasR Fusion Proteins

As used herein, the term fusion protein generally refers to a protein that includes protein or protein components derived from more than one parental protein or protein. Generally, a fusion protein is expressed from a fusion gene in which a nucleotide sequence encoding a protein sequence from one protein is appended in frame with, and optionally separated by a linker from, a nucleotide sequence encoding a protein sequence from a different protein. The fusion gene can then be expressed by a recombinant host cell as a single protein.

A domain of a protein, as used herein, generally is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane, or extracellular domain).

Provided herein are fusion protein comprising a FasR protein and a second polypeptide (e.g., a variant Fc protein). In come embodiments, the second polypeptide extends the half-life of the FasR protein (e.g., an Fc or albumin protein). In some embodiments, the second polypeptide is an Fc protein. In certain embodiments, the Fc protein is a variant Fc protein comprising one or more mutations relative to a wildtype Fc region, wherein the variant Fc region exhibits reduced effector function compared to the wildtype Fc regions. In some embodiments, the second protein comprises any one of SEQ ID NOs: 3-4. In some embodiments, the second protein comprises any one of SEQ ID NOs: 5-6.

In some embodiments, the FasR fusion protein comprises an amino acid sequence having about 80% sequence identity to SEQ ID NO: 6 to about 100% sequence identity to SEQ ID NO: 6. In some embodiments, the FasR fusion protein comprises an amino acid sequence having about 80% sequence identity to SEQ ID NO: 6 to about 85% sequence identity to SEQ ID NO: 6, about 80% sequence identity to SEQ ID NO: 6 to about 90% sequence identity to SEQ ID NO: 6, about 80% sequence identity to SEQ ID NO: 6 to about 95% sequence identity to SEQ ID NO: 6, about 80% sequence identity to SEQ ID NO: 6 to about 97% sequence identity to SEQ ID NO: 6, about 80% sequence identity to SEQ ID NO: 6 to about 98% sequence identity to SEQ ID NO: 6, about 80% sequence identity to SEQ ID NO: 6 to about 99% sequence identity to SEQ ID NO: 6, about 80% sequence identity to SEQ ID NO: 6 to about 100% sequence identity to SEQ ID NO: 6, about 85% sequence identity to SEQ ID NO: 6 to about 90% sequence identity to SEQ ID NO: 6, about 85% sequence identity to SEQ ID NO: 6 to about 95% sequence identity to SEQ ID NO: 6, about 85% sequence identity to SEQ ID NO: 6 to about 97% sequence identity to SEQ ID NO: 6, about 85% sequence identity to SEQ ID NO: 6 to about 98% sequence identity to SEQ ID NO: 6, about 85% sequence identity to SEQ ID NO: 6 to about 99% sequence identity to SEQ ID NO: 6, about 85% sequence identity to SEQ ID NO: 6 to about 100% sequence identity to SEQ ID NO: 6, about 90% sequence identity to SEQ ID NO: 6 to about 95% sequence identity to SEQ ID NO: 6, about 90% sequence identity to SEQ ID NO: 6 to about 97% sequence identity to SEQ ID NO: 6, about 90% sequence identity to SEQ ID NO: 6 to about 98% sequence identity to SEQ ID NO: 6, about 90% sequence identity to SEQ ID NO: 6 to about 99% sequence identity to SEQ ID NO: 6, about 90% sequence identity to SEQ ID NO: 6 to about 100% sequence identity to SEQ ID NO: 6, about 95% sequence identity to SEQ ID NO: 6 to about 97% sequence identity to SEQ ID NO: 6, about 95% sequence identity to SEQ ID NO: 6 to about 98% sequence identity to SEQ ID NO: 6, about 95% sequence identity to SEQ ID NO: 6 to about 99% sequence identity to SEQ ID NO: 6, about 95% sequence identity to SEQ ID NO: 6 to about 100% sequence identity to SEQ ID NO: 6, about 97% sequence identity to SEQ ID NO: 6 to about 98% sequence identity to SEQ ID NO: 6, about 97% sequence identity to SEQ ID NO: 6 to about 99% sequence identity to SEQ ID NO: 6, about 97% sequence identity to SEQ ID NO: 6 to about 100% sequence identity to SEQ ID NO: 6, about 98% sequence identity to SEQ ID NO: 6 to about 99% sequence identity to SEQ ID NO: 6, about 98% sequence identity to SEQ ID NO: 6 to about 100% sequence identity to SEQ ID NO: 6, or about 99% sequence identity to SEQ ID NO: 6 to about 100% sequence identity to SEQ ID NO: 6. In some embodiments, the FasR fusion protein comprises an amino acid sequence having about 80% sequence identity to SEQ ID NO: 6, about 85% sequence identity to SEQ ID NO: 6, about 90% sequence identity to SEQ ID NO: 6, about 95% sequence identity to SEQ ID NO: 6, about 97% sequence identity to SEQ ID NO: 6, about 98% sequence identity to SEQ ID NO: 6, about 99% sequence identity to SEQ ID NO: 6, or about 100% sequence identity to SEQ ID NO: 6. In some embodiments, the FasR fusion protein comprises an amino acid sequence having at least about 80% sequence identity to SEQ ID NO: 6, about 85% sequence identity to SEQ ID NO: 6, about 90% sequence identity to SEQ ID NO: 6, about 95% sequence identity to SEQ ID NO: 6, about 97% sequence identity to SEQ ID NO: 6, about 98% sequence identity to SEQ ID NO: 6, or about 99% sequence identity to SEQ ID NO: 6.

Provided herein are fusion protein comprising a FasR protein and a variant FC protein, wherein the variant Fc protein comprises one or more mutations relative to a wildtype Fc region, wherein the variant Fc region exhibits reduced effector function compared to the wildtype Fc regions. In some embodiments, the FasR fusion protein comprises an amino acid sequence having about 80% sequence identity to SEQ ID NO: 5 to about 100% sequence identity to SEQ ID NO: 5. In some embodiments, the FasR fusion protein comprises an amino acid sequence having about 80% sequence identity to SEQ ID NO: 5 to about 85% sequence identity to SEQ ID NO: 5, about 80% sequence identity to SEQ ID NO: 5 to about 90% sequence identity to SEQ ID NO: 5, about 80% sequence identity to SEQ ID NO: 5 to about 95% sequence identity to SEQ ID NO: 5, about 80% sequence identity to SEQ ID NO: 5 to about 97% sequence identity to SEQ ID NO: 5, about 80% sequence identity to SEQ ID NO: 5 to about 98% sequence identity to SEQ ID NO: 5, about 80% sequence identity to SEQ ID NO: 5 to about 99% sequence identity to SEQ ID NO: 5, about 80% sequence identity to SEQ ID NO: 5 to about 100% sequence identity to SEQ ID NO: 5, about 85% sequence identity to SEQ ID NO: 5 to about 90% sequence identity to SEQ ID NO: 5, about 85% sequence identity to SEQ ID NO: 5 to about 95% sequence identity to SEQ ID NO: 5, about 85% sequence identity to SEQ ID NO: 5 to about 97% sequence identity to SEQ ID NO: 5, about 85% sequence identity to SEQ ID NO: 5 to about 98% sequence identity to SEQ ID NO: 5, about 85% sequence identity to SEQ ID NO: 5 to about 99% sequence identity to SEQ ID NO: 5, about 85% sequence identity to SEQ ID NO: 5 to about 100% sequence identity to SEQ ID NO: 5, about 90% sequence identity to SEQ ID NO: 5 to about 95% sequence identity to SEQ ID NO: 5, about 90% sequence identity to SEQ ID NO: 5 to about 97% sequence identity to SEQ ID NO: 5, about 90% sequence identity to SEQ ID NO: 5 to about 98% sequence identity to SEQ ID NO: 5, about 90% sequence identity to SEQ ID NO: 5 to about 99% sequence identity to SEQ ID NO: 5, about 90% sequence identity to SEQ ID NO: 5 to about 100% sequence identity to SEQ ID NO: 5, about 95% sequence identity to SEQ ID NO: 5 to about 97% sequence identity to SEQ ID NO: 5, about 95% sequence identity to SEQ ID NO: 5 to about 98% sequence identity to SEQ ID NO: 5, about 95% sequence identity to SEQ ID NO: 5 to about 99% sequence identity to SEQ ID NO: 5, about 95% sequence identity to SEQ ID NO: 5 to about 100% sequence identity to SEQ ID NO: 5, about 97% sequence identity to SEQ ID NO: 5 to about 98% sequence identity to SEQ ID NO: 5, about 97% sequence identity to SEQ ID NO: 5 to about 99% sequence identity to SEQ ID NO: 5, about 97% sequence identity to SEQ ID NO: 5 to about 100% sequence identity to SEQ ID NO: 5, about 98% sequence identity to SEQ ID NO: 5 to about 99% sequence identity to SEQ ID NO: 5, about 98% sequence identity to SEQ ID NO: 5 to about 100% sequence identity to SEQ ID NO: 5, or about 99% sequence identity to SEQ ID NO: 5 to about 100% sequence identity to SEQ ID NO: 5. In some embodiments, the FasR fusion protein comprises an amino acid sequence having about 80% sequence identity to SEQ ID NO: 5, about 85% sequence identity to SEQ ID NO: 5, about 90% sequence identity to SEQ ID NO: 5, about 95% sequence identity to SEQ ID NO: 5, about 97% sequence identity to SEQ ID NO: 5, about 98% sequence identity to SEQ ID NO: 5, about 99% sequence identity to SEQ ID NO: 5, or about 100% sequence identity to SEQ ID NO: 5. In some embodiments, the FasR fusion protein comprises an amino acid sequence having at least about 80% sequence identity to SEQ ID NO: 5, about 85% sequence identity to SEQ ID NO: 5, about 90% sequence identity to SEQ ID NO: 5, about 95% sequence identity to SEQ ID NO: 5, about 97% sequence identity to SEQ ID NO: 5, about 98% sequence identity to SEQ ID NO: 5, or about 99% sequence identity to SEQ ID NO: 5.

### Dead Fc Proteins

An Fc region generally encompasses and/or refers to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

Effector functions generally include and/or refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation. In some embodiments, the antibodies described herein comprise mutations that reduce effector function.

In some embodiments, one or more amino acid modifications are introduced into the Fragment crystallizable (Fc) region of a human or humanized antibody, thereby generating an Fc region variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions.

In some embodiments, a variant Fc region comprises at least one amino acid modification in the Fc region. Combining amino acid modifications are also useful. For example, the variant Fc region can include two, three, four, five, etc. substitutions therein, e.g., of the specific Fc region positions identified herein.

In some embodiments, the fusion proteins comprising an Fc protein described herein have a reduced effector function as compared to a human IgG, which in certain instances, is achieved through the use of a dead Fc protein (also referred to as an Fc variant). Effector functions generally refer to a biological event resulting from the interaction of an antibody Fc region with an Fc receptor or ligand. Non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation. In some cases, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some cases, complement dependent cytotoxicity (CDC) refers to lysing of a target cells in the presence of complement, where the complement action pathway is initiated by the binding of C1q to antibody bound with the target.

In certain cases, it is beneficial to reduce the effector function of the antibodies described herein. In some instances, modifications in the Fc region generate an Fc variant with (a) decreased antibody-dependent cell-mediated cytotoxicity ADCC), (b) decreased complement mediated cytotoxicity (CDC), and/or (c) decreased affinity for C1q. In some embodiments, the Fc region is modified to decrease antibody dependent cellular cytotoxicity (ADCC), decrease antibody-dependent cell-mediated phagocytosis (ADCP), decrease complement mediated cytotoxicity (CDC), and/or decrease affinity for C1q by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439 (EU numbering). In some embodiments, the variant Fc region is selected from one or more of the following:

TABLE 1
Dead/Variant Fc Mutations

| Variant Fc Regions and Mutation(s) | Exemplary Effect |
|---|---|
| E233P | Decreases binding to FcγRI, II, III |
| S228P, L235E SPLE in IgG4 | Decreases binding to FcγRI |
| L235E | Decreases binding to FcγRs |
| L234A, L235A | Decreases binding to FcγRI, II, III |
| L234A, L235A, G237A | Decreases binding to FcγRI, II, III, C1q |
| L234A, L235A, P329G | Decreases binding to FcγRI, II, III, C1q |
| L234F, L235E, P331S | Decreases binding to FcγRI, II, III, C1q |
| L234A, L235E, G237A | Decreases binding to FcγRI, II, III, C1q |
| L234A, L235E, G237A, P331S | Decreases binding to FcγRI, II, III, C1q |
| L234A, L235E, G237A, A330S, P331S | Decreases binding to FcγRI, II, III, C1q |
| L234A, L235A, G237A, P238S, H268A, | Decreases binding to FcγRI, IIa, IIb, IIIa |
| A330S, P331S (IgG1-sigma) | |
| L234A, L235A, P329A | Decreases binding to FcγRI, II, III, C1q |
| G236R, L328R | Decreases binding to FcγRI, II, III |
| G237A | Decreases binding to FcγRII |
| F241A | Decreases binding to C1q |
| V264A | Decreases binding to C1q |
| D265A | Decreases binding to FcγRI, II, III |
| D265A, N297A | Decreases binding to FcγRI, II, III, C1q |
| D265A, N297G | Decreases binding to FcγRI, II, III, C1q |
| D270A | Decreases binding to C1q |
| N297A, G, D, Q | Elimination of N-linked glycosylation |
| | Decreases binding to FcγRI, II, III, C1q |
| P329A, G, R | Decreases binding to C1q |
| A330L | Decreases binding to C1q |
| P331A or P331S | Diminished C1q binding |
| S228P | Prevent IgG4 Fab arm exchange |
| S228P, F234A, L235A (IgG4) | Decreases binding to FcγRI, IIa, IIIa |
| H268Q, V309L, A330S, P331S (IgG2m4) | Decreases binding to FcγRI, II, III, C1q |
| V234A, G237A, P238S, H268A, V309L, | Decreases binding to FcγRI, IIa, IIb, IIIa, C1q |
| A330S, P331S (IgG2-sigma) | |
| K322A | Decreases binding to C1q |
| N297A or N297Q or N297G | Reduced FcγR and C1q binding |
| S239D, I332E | Reduced FcγR and C1q binding |
| H268Q, V309L, A330S, P331S | Reduced FcγR and C1q binding |
| V234A, G237A, P238S, H268A, V309L, | Reduced FcγR and C1q binding |
| A330S, P331S | |
| High mannose glycosylation | Decreases binding to C1q |

Provided herein are FasR fusion proteins comprising a FasR protein and a variant Fc selected from Table 1 (e.g., within any row or a combination of rows).

Non-limiting examples of in vitro assays to assess ADCC activity of a molecule of interest is described in U.S. Pat. Nos. 5,500,362 and 5,821,337. Alternatively, non-radioactive assays methods may be employed. Useful effector cells for such assays include peripheral blood mononuclear cells (PBMC), monocytes, macrophages, and Natural Killer (NK) cells.

In some embodiments, variant Fc regions exhibit ADCC that is reduced by at least about 30%, at least about 40%, at least about 50%, at least about 60%, at least about 70% or more as compared to an antibody comprising a non-variant Fc region, e.g., an antibody with the same sequence identity but for the substitution(s) that decrease ADCC (e.g., human IgG1). In some embodiments, variant Fc regions exhibit CDC that is reduced by at least about 30%, at least about 40%, at least about 50%, at least about 60%, at least about 70% or more as compared to an antibody comprising a non-variant Fc region, e.g., an antibody with the same sequence identity but for the substitution(s) that decrease CDC (e.g., human IgG1).

In certain embodiments, variant Fc regions exhibit ADCC that is reduced by about 2% to about 100%. In certain embodiments, variant Fc regions exhibit ADCC that is reduced by about 2% to about 20%, about 2% to about 30%, about 2% to about 40%, about 2% to about 50%, about 2% to about 60%, about 2% to about 70%, about 2% to about 80%, about 2% to about 90%, about 2% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In certain embodiments, variant Fc regions exhibit ADCC that is reduced by about 2%, about 20%, about 30%, about 40%, about 50%, about 60%, about 70%, about 80%, about 90%, or about 100%. In certain embodiments, variant Fc regions exhibit ADCC that is reduced by at least about 2%, about 20%, about 30%, about 40%, about 50%, about 60%, about 70%, about 80%, or about 90%.

In certain embodiments, variant Fc regions exhibit CDC that is reduced by about 2% to about 100%. In certain embodiments, variant Fc regions exhibit CDC that is reduced by about 2% to about 20%, about 2% to about 30%, about 2% to about 40%, about 2% to about 50%, about 2% to about 60%, about 2% to about 70%, about 2% to about 80%, about 2% to about 90%, about 2% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In certain embodiments, variant Fc regions exhibit CDC that is reduced by about 2%, about 20%, about 30%, about 40%, about 50%, about 60%, about 70%, about 80%, about 90%, or about 100%. In certain embodiments, variant Fc regions exhibit CDC that is reduced by at least about 2%, about 20%, about 30%, about 40%, about 50%, about 60%, about 70%, about 80%, or about 90%.

In some embodiments, variant Fc regions exhibit reduced effector function as compared with wild-type human IgG1. Non-limiting examples of Fc mutations in IgG1 that, in certain instances, reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by EU.

In some embodiments, the variant Fc region comprises an IgG1 Fc region comprising an N297A substitution, according to the EU numbering system. In some embodiments, the variant Fc region comprises an IgG1 Fc region comprising an N297Q substitution, according to the EU numbering system. In some embodiments, the variant Fc region comprises an IgG1 Fc region comprising an N297D substitution, according to the EU numbering system. In some embodiments, the variant Fc region comprises an IgG1 Fc region comprising an D265A substitution, according to the EU numbering system. In some embodiments, the variant Fc region comprises an IgG1 Fc region comprising an S228P substitution, according to the EU numbering system. In some embodiments, the variant Fc region comprises an IgG1 Fc region comprising an L235A substitution, according to the EU numbering system. In some embodiments, the variant Fc region comprises an IgG1 Fc region comprising an L237A substitution, according to the EU numbering system. In some embodiments, the variant Fc region comprises an IgG1 Fc region comprising an L234A substitution, according to the EU numbering system. In some embodiments, the variant Fc region comprises an IgG1 Fc region comprising an E233P substitution, according to the EU numbering system. In some embodiments, the variant Fc region comprises an IgG1 Fc region comprising an L234V substitution, according to the EU numbering system. In some embodiments, the variant Fc region comprises an IgG1 Fc region comprising an C236 deletion, according to the EU numbering system. In some embodiments, the variant Fc region comprises an IgG1 Fc region comprising a P238A substitution, according to the EU numbering system. In some embodiments, the variant Fc region comprises an IgG1 Fc region comprising an A327Q substitution, according to the EU numbering system. In some embodiments, the variant Fc region comprises an IgG1 Fc region comprising a P329A substitution, according to the EU numbering system. In some embodiments, the variant Fc region comprises an IgG1 Fc region comprising an P329G substitution, according to the EU numbering system. In some embodiments, the variant Fc region comprises an IgG1 Fc region comprising an L235E substitution, according to the EU numbering system. In some embodiments, the variant Fc region comprises an IgG1 Fc region comprising an P331S substitution, according to the EU numbering system. In some embodiments, the variant Fc region comprises an IgG1 Fc region comprising an L234F substitution, according to the EU numbering system. In some embodiments, the variant Fc region comprises an IgG1 Fc region comprising a 235G substitution, according to the EU numbering system. In some embodiments, the variant Fc region comprises an IgG1 Fc region comprising an 235Q substitution, according to the EU numbering system. In some embodiments, the variant Fc region comprises an IgG1 Fc region comprising an 235R substitution, according to the EU numbering system. In some embodiments, the variant Fc region comprises an IgG1 Fc region comprising an 235S substitution, according to the EU numbering system. In some embodiments, the variant Fc region comprises an IgG1 Fc region comprising an 236F substitution, according to the EU numbering system. In some embodiments, the variant Fc region comprises an IgG1 Fc region comprising an 236R substitution, according to the EU numbering system. In some embodiments, the variant Fc region comprises an IgG1 Fc region comprising an 237E substitution, according to the EU numbering system. In some embodiments, the variant Fc region comprises an IgG1 Fc region comprising an 237K substitution, according to the EU numbering system. In some embodiments, the variant Fc region comprises an IgG1 Fc region comprising an 237N substitution, according to the EU numbering system. In some embodiments, the variant Fc region comprises an IgG1 Fc region comprising an 237R substitution, according to the EU numbering system. In some embodiments, the variant Fc region comprises an IgG1 Fc region comprising an 238A substitution, according to the EU numbering system. In some embodiments, the variant Fc region comprises an IgG1 Fc region comprising an 238E substitution, according to the EU numbering system. In some embodiments, the variant Fc region comprises an IgG1 Fc region comprising an 238G substitution, according to the EU numbering system. In some embodiments, the variant Fc region comprises an IgG1 Fc region comprising an 238H substitution, according to the EU numbering system. In some embodiments, the variant Fc region comprises an IgG1 Fc region comprising an 238I substitution, according to the EU numbering system. In some embodiments, the variant Fc region comprises an IgG1 Fc region comprising an 238V substitution, according to the EU numbering system. In some embodiments, the variant Fc region comprises an IgG1 Fc region comprising an 238W substitution, according to the EU numbering system. In some embodiments, the variant Fc region comprises an IgG1 Fc region comprising an 238Y substitution, according to the EU numbering system. In some embodiments, the variant Fc region comprises an IgG1 Fc region comprising an 248A substitution, according to the EU numbering system. In some embodiments, the variant Fc region comprises an IgG1 Fc region comprising an 254D substitution, according to the EU numbering system. In some embodiments, the variant Fc region comprises an IgG1 Fc region comprising an 254E substitution, according to the EU numbering system. In some embodiments, the variant Fc region comprises an IgG1 Fc region comprising an 254G substitution, according to the EU numbering system. In some embodiments, the variant Fc region comprises an IgG1 Fc region comprising an 254H substitution, according to the EU numbering system. In some embodiments, the variant Fc region comprises an IgG1 Fc region comprising an 254I substitution, according to the EU numbering system. In some embodiments, the variant Fc region comprises an IgG1 Fc region comprising an 254N substitution, according to the EU numbering system. In some embodiments, the variant Fc region comprises an IgG1 Fc region comprising an 254P substitution, according to the EU numbering system. In some embodiments, the variant Fc region comprises an IgG1 Fc region comprising an 254Q substitution, according to the EU numbering system. In some embodiments, the variant Fc region comprises an IgG1 Fc region comprising an 254T substitution, according to the EU numbering system. In some embodiments, the variant Fc region comprises an IgG1 Fc region comprising an 254V substitution, according to the EU numbering system. In some embodiments, the variant Fc region comprises an IgG1 Fc region comprising an 255N substitution, according to the EU numbering system. In some embodiments, the variant Fc region comprises an IgG1 Fc region comprising an 256H substitution, according to the EU numbering system. In some embodiments, the variant Fc region comprises an IgG1 Fc region comprising an 256K substitution, according to the EU numbering system. In some embodiments, the variant Fc region comprises an IgG1 Fc region comprising an 256R substitution, according to the EU numbering system. In some embodiments, the variant Fc region comprises an IgG1 Fc region comprising an 256V substitution, according to the EU numbering system. In some embodiments, the variant Fc region comprises an IgG1 Fc region comprising an 264S substitution, according to the EU numbering system. In some embodiments, the variant Fc region comprises an IgG1 Fc region comprising an 265H substitution, according to the EU numbering system. In some embodiments, the variant Fc region comprises an IgG1 Fc region comprising an 265K substitution, according to the EU numbering system. In some embodiments, the variant Fc region comprises an IgG1 Fc region comprising an 265S substitution, according to the EU numbering system. In some embodiments, the variant Fc region comprises an IgG1 Fc region comprising an 265Y substitution, according to the EU numbering system. In some embodiments, the variant Fc region comprises an IgG1 Fc region comprising an 267G substitution, according to the EU numbering system. In some embodiments, the variant Fc region comprises an IgG1 Fc region comprising an 267H substitution, according to the EU numbering system. In some embodiments, the variant Fc region comprises an IgG1 Fc region comprising an 267I substitution, according to the EU numbering system. In some embodiments, the variant Fc region comprises an IgG1 Fc region comprising an 267K substitution, according to the EU numbering system. In some embodiments, the variant Fc region comprises an IgG1 Fc region comprising an 268K substitution, according to the EU numbering system. In some embodiments, the variant Fc region comprises an IgG1 Fc region comprising an 269N substitution, according to the EU numbering system. In some embodiments, the variant Fc region comprises an IgG1 Fc region comprising an 269Q substitution, according to the EU numbering system. In some embodiments, the variant Fc region comprises an IgG1 Fc region comprising an 270A substitution, according to the EU numbering system. In some embodiments, the variant Fc region comprises an IgG1 Fc region comprising an 270G substitution, according to the EU numbering system. In some embodiments, the variant Fc region comprises an IgG1 Fc region comprising an 270M substitution, according to the EU numbering system. In some embodiments, the variant Fc region comprises an IgG1 Fc region comprising an 270N substitution, according to the EU numbering system. In some embodiments, the variant Fc region comprises an IgG1 Fc region comprising an 271T substitution, according to the EU numbering system. In some embodiments, the variant Fc region comprises an IgG1 Fc region comprising an 272N substitution, according to the EU numbering system. In some embodiments, the variant Fc region comprises an IgG1 Fc region comprising an 279F substitution, according to the EU numbering system. In some embodiments, the variant Fc region comprises an IgG1 Fc region comprising an 279K substitution, according to the EU numbering system. In some embodiments, the variant Fc region comprises an IgG1 Fc region comprising an 279L substitution, according to the EU numbering system. In some embodiments, the variant Fc region comprises an IgG1 Fc region comprising an 292E substitution, according to the EU numbering system. In some embodiments, the variant Fc region comprises an IgG1 Fc region comprising an 292F substitution, according to the EU numbering system. In some embodiments, the variant Fc region comprises an IgG1 Fc region comprising an 292G substitution, according to the EU numbering system. In some embodiments, the variant Fc region comprises an IgG1 Fc region comprising an 292I substitution, according to the EU numbering system. In some embodiments, the variant Fc region comprises an IgG1 Fc region comprising an 293S substitution, according to the EU numbering system. In some embodiments, the variant Fc region comprises an IgG1 Fc region comprising an 301W substitution, according to the EU numbering system. In some embodiments, the variant Fc region comprises an IgG1 Fc region comprising an 304E substitution, according to the EU numbering system. In some embodiments, the variant Fc region comprises an IgG1 Fc region comprising an 311E substitution, according to the EU numbering system. In some embodiments, the variant Fc region comprises an IgG1 Fc region comprising an 311G substitution, according to the EU numbering system. In some embodiments, the variant Fc region comprises an IgG1 Fc region comprising an 311S substitution, according to the EU numbering system. In some embodiments, the variant Fc region comprises an IgG1 Fc region comprising an 316F substitution, according to the EU numbering system. In some embodiments, the variant Fc region comprises an IgG1 Fc region comprising an 327T substitution, according to the EU numbering system. In some embodiments, the variant Fc region comprises an IgG1 Fc region comprising an 328V substitution, according to the EU numbering system. In some embodiments, the variant Fc region comprises an IgG1 Fc region comprising an 329Y substitution, according to the EU numbering system. In some embodiments, the variant Fc region comprises an IgG1 Fc region comprising an 330R substitution, according to the EU numbering system. In some embodiments, the variant Fc region comprises an IgG1 Fc region comprising an 339E substitution, according to the EU numbering system. In some embodiments, the variant Fc region comprises an IgG1 Fc region comprising an 339L substitution, according to the EU numbering system. In some embodiments, the variant Fc region comprises an IgG1 Fc region comprising an 343I substitution, according to the EU numbering system. In some embodiments, the variant Fc region comprises an IgG1 Fc region comprising an 343V substitution, according to the EU numbering system. In some embodiments, the variant Fc region comprises an IgG1 Fc region comprising an 373A substitution, according to the EU numbering system. In some embodiments, the variant Fc region comprises an IgG1 Fc region comprising an 373G substitution, according to the EU numbering system. In some embodiments, the variant Fc region comprises an IgG1 Fc region comprising an 373S substitution, according to the EU numbering system. In some embodiments, the variant Fc region comprises an IgG1 Fc region comprising an 376E substitution, according to the EU numbering system. In some embodiments, the variant Fc region comprises an IgG1 Fc region comprising an 376W substitution, according to the EU numbering system. In some embodiments, the variant Fc region comprises an IgG1 Fc region comprising an 376Y substitution, according to the EU numbering system. In some embodiments, the variant Fc region comprises an IgG1 Fc region comprising an 380D substitution, according to the EU numbering system. In some embodiments, the variant Fc region comprises an IgG1 Fc region comprising an 382D substitution, according to the EU numbering system. In some embodiments, the variant Fc region comprises an IgG1 Fc region comprising an 382P substitution, according to the EU numbering system. In some embodiments, the variant Fc region comprises an IgG1 Fc region comprising an 385P substitution, according to the EU numbering system. In some embodiments, the variant Fc region comprises an IgG1 Fc region comprising an 424H substitution, according to the EU numbering system. In some embodiments, the variant Fc region comprises an IgG1 Fc region comprising an 424M substitution, according to the EU numbering system. In some embodiments, the variant Fc region comprises an IgG1 Fc region comprising an 424V substitution, according to the EU numbering system. In some embodiments, the variant Fc region comprises an IgG1 Fc region comprising an 434I substitution, according to the EU numbering system. In some embodiments, the variant Fc region comprises an IgG1 Fc region comprising an 438G substitution, according to the EU numbering system. In some embodiments, the variant Fc region comprises an IgG1 Fc region comprising an 439E substitution, according to the EU numbering system. In some embodiments, the variant Fc region comprises an IgG1 Fc region comprising an 439H substitution, according to the EU numbering system. In some embodiments, the variant Fc region comprises an IgG1 Fc region comprising an 439Q substitution, according to the EU numbering system. In some embodiments, the variant Fc region comprises an IgG1 Fc region comprising an 440A substitution, according to the EU numbering system. In some embodiments, the variant Fc region comprises an IgG1 Fc region comprising an 440D substitution, according to the EU numbering system. In some embodiments, the variant Fc region comprises an IgG1 Fc region comprising an 440E substitution, according to the EU numbering system. In some embodiments, the variant Fc region comprises an IgG1 Fc region comprising an 440F substitution, according to the EU numbering system. In some embodiments, the variant Fc region comprises an IgG1 Fc region comprising an 440M substitution, according to the EU numbering system. In some embodiments, the variant Fc region comprises an IgG1 Fc region comprising an 440T Fc region substitution, according to the EU numbering system. In some embodiments, the variant Fc region comprises an IgG1 Fc region comprising an 440V substitution, according to the EU numbering system.

In some embodiments, the variant Fc region comprises an IgG1 Fc region L234A, L235E, G237A, A330S, and/or P331S by EU Numbering. In some embodiments, the variant Fc region comprises an IgG1 Fc region comprising E233P, according to the EU numbering system. In some embodiments, the variant Fc region comprises an IgG4 Fc region comprising S228P and L235E. In some embodiments, the variant Fc region comprises an IgG1 Fc region comprising L235E, according to the EU numbering system. In some embodiments, the variant Fc region comprises an IgG1 Fc region comprising L234A and L235A, according to the EU numbering system. In some embodiments, the variant Fc region comprises an IgG1 Fc region comprising L234A, L235A, and G237A, according to the EU numbering system. In some embodiments, the variant Fc region comprises an IgG1 Fc region comprising L234A, L235A, P329G, according to the EU numbering system. In some embodiments, the variant Fc region comprises an IgG1 Fc region comprising L234F, L235E, and P331S, according to the EU numbering system. In some embodiments, the variant Fc region comprises an IgG1 Fc region comprising L234A, L235E, and G237A, according to the EU numbering system. In some embodiments, the variant Fc region comprises an IgG1 Fc region comprising L234A, L235E, G237A, and P331S, according to the EU numbering system. In some embodiments, the variant Fc region comprises an IgG1 Fc region comprising L234A, L235A, G237A, P238S, H268A, A330S, and P331S (IgG1), according to the EU numbering system. In some embodiments, the variant Fc region comprises an IgG1 Fc region comprising L234A, L235A, and P329A, according to the EU numbering system. In some embodiments, the variant Fc region comprises an IgG1 Fc region comprising G236R and L328R, according to the EU numbering system. In some embodiments, the variant Fc region comprises an IgG1 Fc region comprising G237A, according to the EU numbering system. In some embodiments, the variant Fc region comprises an IgG1 Fc region comprising F241A, according to the EU numbering system. In some embodiments, the variant Fc region comprises an IgG1 Fc region comprising V264A, according to the EU numbering system. In some embodiments, the variant Fc region comprises an IgG1 Fc region comprising D265A, according to the EU numbering system. In some embodiments, the variant Fc region comprises an IgG1 Fc region comprising D265A and N297A, according to the EU numbering system. In some embodiments, the variant Fc region comprises an IgG1 Fc region comprising D265A and N297G, according to the EU numbering system. In some embodiments, the variant Fc region comprises an IgG1 Fc region comprising D270A, according to the EU numbering system. In some embodiments, the variant Fc region comprises an IgG1 Fc region comprising N297A, according to the EU numbering system. In some embodiments, the variant Fc region comprises an IgG1 Fc region comprising N297G, according to the EU numbering system. In some embodiments, the variant Fc region comprises an IgG1 Fc region comprising N297D, according to the EU numbering system. In some embodiments, the variant Fc region comprises an IgG1 Fc region comprising N297Q, according to the EU numbering system. In some embodiments, the variant Fc region comprises an IgG1 Fc region comprising P329A, according to the EU numbering system. In some embodiments, the variant Fc region comprises an IgG1 Fc region comprising P329G, according to the EU numbering system. In some embodiments, the variant Fc region comprises an IgG1 Fc region comprising P329R, according to the EU numbering system. In some embodiments, the variant Fc region comprises an IgG1 Fc region comprising A330L, according to the EU numbering system. In some embodiments, the variant Fc region comprises an IgG1 Fc region comprising P331A, according to the EU numbering system. In some embodiments, the variant Fc region comprises an IgG1 Fc region comprising P331S, according to the EU numbering system. In some embodiments, the variant Fc region comprises an IgG2 Fc region. In some embodiments, the variant Fc region comprises an IgG4 Fc region. In some embodiments, the variant Fc region comprises an IgG4 Fc region comprising S228P, according to the EU numbering system. In some embodiments, the variant Fc region comprises an IgG4 Fc region comprising S228P, F234A, and L235A, according to the EU numbering system. In some embodiments, the variant Fc region comprises an IgG2-IgG4 cross-subclass (IgG2/G4) Fc region. In some embodiments, the variant Fc region comprises an IgG2-IgG3 cross-subclass Fc region. In some embodiments, the variant Fc region comprises an IgG2 Fc region comprising H268Q, V309L, A330S, and P331S, according to the EU numbering system. In some embodiments, the variant Fc region comprises an IgG2 Fc region comprising V234A, G237A, P238S, H268A, V309L, A330S, and P331S, according to the EU numbering system. In some embodiments, an antibody comprises a Fc region comprising high mannose glycosylation.

In some embodiments, the variant Fc region comprises an IgG4 Fc region comprising a S228P substitution, according to the EU numbering system. In some embodiments, the variant Fc region comprises an IgG4 Fc region comprising an A330S substitution, according to the EU numbering system. In some embodiments, the variant Fc region comprises an IgG4 Fc region comprising a P331S substitution, according to the EU numbering system.

In some embodiments, the variant Fc region comprises an IgG2 Fc region comprising an A330S substitution, according to the EU numbering system. In some embodiments, the variant Fc region comprises an IgG2 Fc region comprising an P331S substitution, according to the EU numbering system. In some embodiments, the variant Fc region comprises an IgG2 Fc region comprising an 234A substitution, according to the EU numbering system. In some embodiments, the variant Fc region comprises an IgG2 Fc region comprising an 237A substitution, according to the EU numbering system.

In some embodiments, the variant Fc region comprises IgG1 Fc region, and wherein the one or more mutations comprises (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (I) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(uu), per EU numbering. In some embodiments, the Variant Fc comprises SEQ ID NO: 3. In some embodiments, the FasR fusion protein comprises SEQ ID NO: 5.

In some embodiments, the FasR fusion protein comprises an amino acid sequence having about 80% sequence identity to SEQ ID NO: 5 to about 100% sequence identity to SEQ ID NO: 5. In some embodiments, the FasR fusion protein comprises an amino acid sequence having about 80% sequence identity to SEQ ID NO: 5 to about 85% sequence identity to SEQ ID NO: 5, about 80% sequence identity to SEQ ID NO: 5 to about 90% sequence identity to SEQ ID NO: 5, about 80% sequence identity to SEQ ID NO: 5 to about 95% sequence identity to SEQ ID NO: 5, about 80% sequence identity to SEQ ID NO: 5 to about 97% sequence identity to SEQ ID NO: 5, about 80% sequence identity to SEQ ID NO: 5 to about 98% sequence identity to SEQ ID NO: 5, about 80% sequence identity to SEQ ID NO: 5 to about 99% sequence identity to SEQ ID NO: 5, about 80% sequence identity to SEQ ID NO: 5 to about 100% sequence identity to SEQ ID NO: 5, about 85% sequence identity to SEQ ID NO: 5 to about 90% sequence identity to SEQ ID NO: 5, about 85% sequence identity to SEQ ID NO: 5 to about 95% sequence identity to SEQ ID NO: 5, about 85% sequence identity to SEQ ID NO: 5 to about 97% sequence identity to SEQ ID NO: 5, about 85% sequence identity to SEQ ID NO: 5 to about 98% sequence identity to SEQ ID NO: 5, about 85% sequence identity to SEQ ID NO: 5 to about 99% sequence identity to SEQ ID NO: 5, about 85% sequence identity to SEQ ID NO: 5 to about 100% sequence identity to SEQ ID NO: 5, about 90% sequence identity to SEQ ID NO: 5 to about 95% sequence identity to SEQ ID NO: 5, about 90% sequence identity to SEQ ID NO: 5 to about 97% sequence identity to SEQ ID NO: 5, about 90% sequence identity to SEQ ID NO: 5 to about 98% sequence identity to SEQ ID NO: 5, about 90% sequence identity to SEQ ID NO: 5 to about 99% sequence identity to SEQ ID NO: 5, about 90% sequence identity to SEQ ID NO: 5 to about 100% sequence identity to SEQ ID NO: 5, about 95% sequence identity to SEQ ID NO: 5 to about 97% sequence identity to SEQ ID NO: 5, about 95% sequence identity to SEQ ID NO: 5 to about 98% sequence identity to SEQ ID NO: 5, about 95% sequence identity to SEQ ID NO: 5 to about 99% sequence identity to SEQ ID NO: 5, about 95% sequence identity to SEQ ID NO: 5 to about 100% sequence identity to SEQ ID NO: 5, about 97% sequence identity to SEQ ID NO: 5 to about 98% sequence identity to SEQ ID NO: 5, about 97% sequence identity to SEQ ID NO: 5 to about 99% sequence identity to SEQ ID NO: 5, about 97% sequence identity to SEQ ID NO: 5 to about 100% sequence identity to SEQ ID NO: 5, about 98% sequence identity to SEQ ID NO: 5 to about 99% sequence identity to SEQ ID NO: 5, about 98% sequence identity to SEQ ID NO: 5 to about 100% sequence identity to SEQ ID NO: 5, or about 99% sequence identity to SEQ ID NO: 5 to about 100% sequence identity to SEQ ID NO: 5. In some embodiments, the FasR fusion protein comprises an amino acid sequence having about 80% sequence identity to SEQ ID NO: 5, about 85% sequence identity to SEQ ID NO: 5, about 90% sequence identity to SEQ ID NO: 5, about 95% sequence identity to SEQ ID NO: 5, about 97% sequence identity to SEQ ID NO: 5, about 98% sequence identity to SEQ ID NO: 5, about 99% sequence identity to SEQ ID NO: 5, or about 100% sequence identity to SEQ ID NO: 5. In some embodiments, the FasR fusion protein comprises an amino acid sequence having at least about 80% sequence identity to SEQ ID NO: 5, about 85% sequence identity to SEQ ID NO: 5, about 90% sequence identity to SEQ ID NO: 5, about 95% sequence identity to SEQ ID NO: 5, about 97% sequence identity to SEQ ID NO: 5, about 98% sequence identity to SEQ ID NO: 5, or about 99% sequence identity to SEQ ID NO: 5.

### Linkers

A protein of the fusion proteins described herein can be “fused” or “linked” via a linker amino acid sequence. The linker may be a protein linker or other linker of suitable flexibility so as not to inhibit binding of either targeting protein (e.g., the IL2 protein or TBRII receptor protein). The linker protein can be unstructured (e.g., lacking secondary structure), structured, or a combination thereof. In some embodiments, the linker sequence is a natural amino acid sequence of a FasR protein or a Fc protein. In some embodiments, the linker is not native to a FasR protein or a Fc protein. For example, a non-native linker can comprise poly-glycine, poly-alanine, poly-serine amino acid or a combination thereof (e.g., GSSG, GGSS, GSAGG, etc.).

In some embodiments, the linker is a non-nature or synthetic linker. The term “synthetic linker” as used herein includes a chemical moiety comprising or derived from a group of atoms that is covalently attached to a targeting agent, and that is also covalently attached to a cytotoxic moiety. Linkers include compounds comprising or derived from divalent radicals such as an alkylene, an arylene, a heteroarylene, moieties for example: -(CR2)nO(CR2)n- wherein R2 is independently repeating units of alkyloxy (e.g. polyethylenoxy, PEG, polymethyleneoxy) and alkylamino (e.g. polyethyleneamino, polyetheramines, for example, Jeffamine™) and n is independently >1, in particular n may be 1 to 15; compounds including the linkers described in Example 1, N-succinimidyl 4-(maleimidomethyl)cyclohexanecarboxylate (SMCC) and N-succinimidyl 4-(2-pyridyldithio)butanoate (SPDB); and diacid ester and amides including succinate, succinamide, diglycolate, malonate, and caproamide as well as peptides, for example, but not limited to repeating units of G, A and C (for example up to 10) with one or more lys residues or other suitable chemical groups for linking to a targeting agent and a cytotoxic moiety. The linker is optionally CI-30 alkylene, unsubstituted or substituted with one or more substituents, and/or optionally interrupted with one or more heteromoieties independently selected from O, S, NR1, and/or optionally interrupted with one or more of C(O) and C(S), wherein RI is independently selected from H, and CI-6 alkyl. The linker can comprise a non-cleavable (stable linker) or cleavable unit (labile linker), for example, a peptide bond or a disulfide bond. The linker can be conjugated to the targeting agent and/or the cytotoxic moiety via reactive functional groups.

Both cleavable and non-cleavable linkers can be used in the synthesis of fusion proteins (ADCs). Cleavable linkers include motifs that are either sensitive to lysosomal proteases or sensitive to an acidic pH (such as hydrazone, which is hydrolysed to cleave the linker in gemtuzumab ozogamicin and inotuzumab ozogamicin), or they can contain disulfide bridges that can be reduced by glutathione. The steric hindrance of disulfide bridges can be optimized to limit premature cleavage inside the cell. Generally, the disulfide linker is initially cleaved to release the thiol compound. Acid-cleavable linkers, for example, hydrazone, are designed to remain stable at the neutral pH in the blood circulation, but in acidic cellular compartments they undergo hydrolysis and release the cytotoxic drug.

In some embodiments, the linker (e.g., peptide linker) comprises about 2 amino acids to about 100 amino acids. In some embodiments, the linker comprises about 2 amino acids to about 15 amino acids, about 2 amino acids to about 20 amino acids, about 2 amino acids to about 25 amino acids, about 2 amino acids to about 30 amino acids, about 2 amino acids to about 40 amino acids, about 2 amino acids to about 50 amino acids, about 2 amino acids to about 75 amino acids, about 2 amino acids to about 100 amino acids, about 15 amino acids to about 20 amino acids, about 15 amino acids to about 25 amino acids, about 15 amino acids to about 30 amino acids, about 15 amino acids to about 40 amino acids, about 15 amino acids to about 50 amino acids, about 15 amino acids to about 75 amino acids, about 15 amino acids to about 100 amino acids, about 20 amino acids to about 25 amino acids, about 20 amino acids to about 30 amino acids, about 20 amino acids to about 40 amino acids, about 20 amino acids to about 50 amino acids, about 20 amino acids to about 75 amino acids, about 20 amino acids to about 100 amino acids, about 25 amino acids to about 30 amino acids, about 25 amino acids to about 40 amino acids, about 25 amino acids to about 50 amino acids, about 25 amino acids to about 75 amino acids, about 25 amino acids to about 100 amino acids, about 30 amino acids to about 40 amino acids, about 30 amino acids to about 50 amino acids, about 30 amino acids to about 75 amino acids, about 30 amino acids to about 100 amino acids, about 40 amino acids to about 50 amino acids, about 40 amino acids to about 75 amino acids, about 40 amino acids to about 100 amino acids, about 50 amino acids to about 75 amino acids, about 50 amino acids to about 100 amino acids, or about 75 amino acids to about 100 amino acids. In some embodiments, the linker comprises about 2 amino acids, about 15 amino acids, about 20 amino acids, about 25 amino acids, about 30 amino acids, about 40 amino acids, about 50 amino acids, about 75 amino acids, or about 100 amino acids. In some embodiments, the linker comprises at least about 2 amino acids, about 15 amino acids, about 20 amino acids, about 25 amino acids, about 30 amino acids, about 40 amino acids, about 50 amino acids, or about 75 amino acids. In some embodiments, the linker comprises at most about 15 amino acids, about 20 amino acids, about 25 amino acids, about 30 amino acids, about 40 amino acids, about 50 amino acids, about 75 amino acids, or about 100 amino acids.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to the eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition; delaying or eliminating the onset of symptoms of a disease or condition; slowing, halting, or reversing the progression of a disease or condition; or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, disorder, or disease, or at least partially inhibit activity of a targeted enzyme or receptor.

The term “immune cell” refers generally to cells of the immune system. Immune cells are derived from myeloid or lymphoid cell linages. Generally, the methods disclosed herein are directed towards, but not limited to, the activation of immune effector cells.

The term “immune effector cell” refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes. In certain embodiments, T cells, NK cells, B cells, NKT cells and gamma/delta T cells or a combination thereof are activated by the fusion protein.

The term “immune effector function” or “immune effector response,” as that term is used herein, refers to function or response, e.g., of an immune effector cell, that enhances or promotes an immune attack to a target cell. E.g., an immune effector function or response refers to a property of a T cells or NK cells that promotes killing or the inhibition of growth or proliferation, of a target cell. Immune effector function includes direct cytotoxicity, cytokine release, chemokine release, phagocytosis, or other immune function that primes or perpetuates an immune response.

The term “effector function” refers to a specialized function of a cell. Effector function of a T cell, for example, may be cytolytic activity or helper activity including the secretion of cytokines.

Throughout this application, various embodiments may be presented in a range formats. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range, for example, from 1 to 6 should be considered to have specifically disclosed subranges, for example, from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. In some embodiments, the subject may be diagnosed or suspected of being at high risk for cancer. In some embodiments, the subject may be diagnosed or suspected of being at high risk for having a tumor. In some embodiments, the subject is not necessarily diagnosed or suspected of being at high risk for cancer.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

### EXAMPLES

### Example 1—FasR Proteins

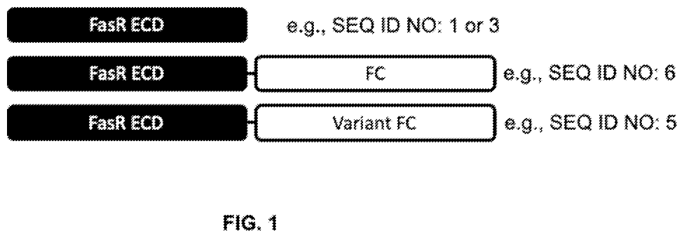

Exemplary FasR proteins and FasR fusion proteins structures are shown in FIG. 1.

SEQUENCES

| NO: | Sequence | Annotation |
|---|---|---|
| 1 | QVTDINSKGLELRKTVTTVETQNLEGLHHDGQFCHKPCPPGERKARDC | FasR |
| | TVNGDEPDCVPCQEGKEYTDKAHFSSKCRRCRLCDEGHGLEVEINCTR | |
| | TQNTKCRCKPNFFCNSTVCEHCDPCTKCEHGIIKECTLTSNTKCKEEG | |
| | SRSN | |
| 2 | AQVTDINSKGLELRKTVTTVETQNLEGLHHDGQFCHKPCPPGERKARD | FasR |
| | CTVNGDEPDCVPCQEGKEYTDKAHFSSKCRRCRLCDEGHGLEVEINCT | |
| | RTQNTKCRCKPNFFCNSTVCEHCDPCTKCEHGIIKECTLTSNTKCKEE | |
| | GSRS | |
| 3 | APEAAGGPSVFLFPPKPKDTLMISRTPEVTCVVVDVSHEDPEVKFNWY | Fc_variant |
| | VDGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNV | |
| | FSCSVMHEALHNHYTQKSLSLSPG | |
| 4 | APELLGGPSVFLFPPKPKDTLMISRTPEVTCVVVDVSHEDPEVKFNWY | Fc |
| | VDGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNV | |
| | FSCSVMHEALHNHYTQKSLSLSPGK | |
| 5 | MVGIWTLLPLVLTSVARLSSKSVNAQVTDINSKGLELRKTVTTVETQN | FL |
| | LEGLHHDGQFCHKPCPPGERKARDCTVNGDEPDCVPCQEGKEYTDKAH | |
| | FSSKCRRCRLCDEGHGLEVEINCTRTQNTKCRCKPNFFCNSTVCEHCD | |
| | PCTKCEHGIIKECTLTSNTKCKEEGSRSCDKTHTCPPCPAPEAAGGPS | |
| | VFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWES | |
| | NGQPENNYKTTPPVLDSDGSFFLYSKLTVDKSRWQQGNVFSCSVMHEA | |
| | LHNHYTQKSLSLSPG | |
| 6 | MVGIWTLLPLVLTSVARLSSKSVNAQVTDINSKGLELRKTVTTVETQN | FL |
| | LEGLHHDGQFCHKPCPPGERKARDCTVNGDEPDCVPCQEGKEYTDKAH | |
| | FSSKCRRCRLCDEGHGLEVEINCTRTQNTKCRCKPNFFCNSTVCEHCD | |
| | PCTKCEHGIIKECTLTSNTKCKEEGSRSCDKTHTCPPCPAPELLGGPS | |
| | VFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKT | |
| | ISKAKGQPREPQVYTLPPSREEMTKNQVSLTCLVKGFYPSDIAVEWES | |
| | NGQPENNYKTTPPVLDSDGSFFLYSKLTVDKSRWQQGNVFSCSVMHEA | |
| | LHNHYTQKSLSLSPGK | |

1. A method of treating retinal cell loss or a symptom thereof in an eye of an individual, the method comprising:
administering a FasR protein to the eye, wherein the FasR protein reduces, inhibits, and/or prevents retinal cell loss and/or the symptom thereof.

2. The method of claim 1, wherein the retinal cells comprise pigment epithelial cells, retinal ganglion cells, photoreceptors, or a combination thereof.

3. The method of claim 1, wherein the FasR protein comprises a FasR monomer, a FasR dimer, or a FasR trimer.

4. The method of claim 1, wherein the FasR protein is fused to a second protein.

5. The method of claim 4, wherein the second protein comprises an Fc protein.

6. A method of treating an ocular disorder or a symptom thereof, in an eye of an individual, the method comprising:
administering a FasR protein to the eye, thereby treating the ocular disorder or symptom thereof.

7. The method of claim 6, wherein the ocular disorder comprises retinal degeneration.

8. The method of claim 6, wherein the ocular disorder comprises glaucoma.

9. The method of claim 6, wherein the ocular disorder comprises macular degeneration.

10. The method of claim 6, wherein the ocular disorder comprises a retinal detachment.

11. The method of claim 6, wherein the ocular disorder comprises inherited retinal degeneration (e.g., retinitis pigmentosa).

12. The method of claim 6, wherein the FasR protein comprises a FasR monomer, a FasR dimer, or a FasR trimer.

13. The method of claim 6, wherein the FasR protein is fused to a second protein.

14. The method of claim 13, wherein the second protein extends the half-life of the FasR protein.

15. The method of claim 13, wherein the second protein comprises an Fc protein.

16. The method of claim 15, wherein the second protein comprises a variant Fc protein comprising one or more mutations relative to a wildtype Fc region, wherein the variant Fc region exhibits reduced effector function compared to the wildtype Fc region.

17. The method of claim 15, wherein the reduced effector function is selected from the list consisting of reduced antibody-dependent cell-mediated cytotoxicity (ADCC), reduced complement mediated cytotoxicity (CDC), reduced affinity for C1q, and any combination thereof.

18. The method of claim 16, wherein the variant Fc protein comprises an IgG1 Fc region, and wherein the one or more mutations comprises (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (I) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(uu), per EU numbering.

19. A method of treating inflammation in retinal tissue and/or a symptom thereof in an eye of an individual, the method comprising:
administering a polynucleic acid molecule comprising a sequence encoding a FasR protein to the eye, wherein the FasR protein reduces, inhibits, and/or prevents inflammation in the retinal tissue and/or the symptom thereof.
